(19) United States
(12) Patent Application Publication
(10) Pub. No.: US 2012/0034470 A1
(43) Pub. Date: Feb. 9, 2012

(54) Low Coefficient of Friction Coating for Metallic Surfaces

(76) Inventors: Kendall L. Guyer, Bartlett, IL (US); Melania Lapinski, Malta, NY (US); Edward F. Landry, Waterford, NY (US); Roy M. Griswold, Ballston Spa, NY (US)

(21) Appl. No.: 13/021,947

(22) Filed: Feb. 7, 2011

(30) Foreign Application Priority Data
Aug. 9, 2010 (KR) 10-2010-0076651

(51) Int. Cl. B32B 15/08; B05D 1/36; B05D 3/00; A61B 17/06; B05D 1/18; B05D 1/28; B05D 3/02; B32B 9/04; B05D 1/02

(52) U.S. Cl. 428/447; 427/331; 427/372.2; 606/223

(57) ABSTRACT

Disclosed herein is an article or device such as surgical devices, needles such as surgical needles, razors, cutting/slicing devices containing a metallic surface, a primer layer disposed on the metallic surface, and a top coat disposed on the primer layer, wherein the primer layer is formed from a primer composition containing a silanol-containing organopolysiloxane resin and a crosslinking agent selected from the group consisting of alkyl silicates, boric acid, borates, acetoxysilanes, ketoximesilanes, and combinations thereof, and wherein the top coat is formed from a top coat composition containing an alkenyl functional polyorganosiloxane, an organohydrogensiloxane, and a hydrosilylation catalyst.

### FIELD

The present invention relates to articles or devices having at least one metallic surface coated with a low coefficient of friction coating. More particularly, it relates to such articles or devices including at least one metallic surface, which is primed with at least partially condensation cured silanol functional siloxane, and then coated with a top coat of addition cured alkenyl functional siloxane. The present invention also relates to methods to prepare such coated articles or devices.

### BACKGROUND

Coating of metallic surfaces of articles such as surgical devices, needles, razors, with silicone compositions is generally known. For example, U.S. Pat. No. 6,210,437 discloses medical devices including a metallic surface coated with a first layer of a short chain silicone having silicon hydroxide and silicon hydride groups, and a second layer of a silicone polymer, wherein the short chain silicone in the first layer is bound to the metal via a silicon hydroxide group that is not at the silicon-hydride terminus, and the silicone polymer in the second layer is covalently bound via the vinyl group to the silicon hydride group of the short chain silicone in the first layer.

U.S. Pat. No. 5,985,355 discloses a process for coating surgical needles. The process includes forming a first leveling coating on the surface of the needle by applying a condensable polymethyl siloxane-containing composition to the surface of the needle, and forming a second slip coating over the leveling coating by applying an amino and alkoxy functional siloxane-containing composition over the first coating.

U.S. Pat. No. 4,720,521 discloses a film-forming siloxane composition for use to coat hypodermic needles, razor blades, catheters and the like. The composition contains a reactive component including a combination of a first siloxane polymer having two or more vinyl groups, a second siloxane crosslinking polymer having two or more pendent hydrogen groups, a third siloxane chain extending polymer having two or more terminal hydrogen groups, and a non-reactive component having a formula of (R)₃SiO(SiR₂O)_{z}Si(R)₃, wherein R is C1-C20 alkyl, haloalkyl, aryl, haloaryl, cycloalkyl, silacyclopentyl, aralkyl and mixtures thereof, and Z is about 20 to about 1,800.

U.S. Pat. No. 6,936,297 discloses methods for making siliconized surgical needles by employing a coating mixture of at least one polydialkylsiloxane and at least one other siliconization material containing an aminoalkyl siloxane and at least one other copolymerizable siloxane.

U.S. Pat. No. 5,536,582 discloses an aqueous silicone coating composition which can be used to lubricate substrates such as suture needles. The coating composition contains a non-reactive polydimethylsiloxane, a reactive siloxane polymer, and at least one dispersing agent, wherein the reactive siloxane polymer is a mixture of an aminoalkyl siloxane and at least one other copolymerizable siloxane.

Unfortunately, some of the coating materials in the prior art require high cure temperatures and long cure cycles. For example, US '437 exemplified that the coatings disclosed therein were cured at 140° C. for 4 hours. US '355 discloses that curing of the condensable polymethyl siloxane takes place for a period in the range of one hour to five hours at a temperature in the range of 130° C. to 250° C. US '297 discloses that the coated needles are placed in a furnace or oven at a temperature of from about 100° C. to 200° C. for a time period ranging from about 2 hours to about 48 hours.

Heretofore, cured coating materials have often been less than satisfactory with respect to their ability to bond cohesively to the metal substrates. Accordingly, the durability of these coatings may be limited due to the risk of delamination from the substrates.

Further, some of the prior art coatings may have a relatively high coefficient of friction. This is not desirable because such coatings are not suitable for applications that demand low friction, such as surgical needles requiring low tissue penetration force, and razor blades requiring low skin irritation.

Accordingly, there is a need in the coatings community for an article or device having a metallic surface that is coated with a durable and low coefficient of friction coating, which can be cured at a lower temperature in a shorter cure period than those of the prior art. The present invention provides an answer to that need.

### SUMMARY

In one aspect, the present invention relates to an article comprising a metallic surface, a primer layer disposed on said metallic surface, and a top coat disposed on said primer layer, wherein said primer layer is formed from a primer composition containing a silanol-containing organopolysiloxane resin and a crosslinking agent selected from the group consisting of alkyl silicates, boric acid, borates, acetoxysilanes, ketoximesilanes, and combinations thereof, and wherein said top coat is formed from a top coat composition containing an alkenyl functional polyorganosiloxane, an organohydrogensiloxane, and a hydrosilylation catalyst.

The article of the invention is suitable for use in applications such as healthcare related devices for example surgical devices, needles such as surgical needles, razors, cutting/slicing devices and articles. The coatings on the articles are durable and exhibit low coefficient of friction. Applying this coating to devices such as surgical needles can help improve the ability of the device to pass through tissue with less force.

In another aspect, the present invention relates to a method for making an article or a device having a coated metallic surface comprising the steps of: (1) providing an article or a device having at least one metallic surface; (2) providing a primer composition and a coating composition, wherein the primer composition contains a silanol-containing organopolysiloxane resin and a crosslinking agent selected from the group consisting of alkyl silicates, boric acid, borates, acetoxysilanes, ketoximesilanes, and combinations thereof, and wherein the top coat composition contains an alkenyl functional polyorganosiloxane, an organohydrogensiloxane, and a hydrosilylation catalyst; (3) contacting said metallic surface with said primer composition to form a primer layer; (4) contacting said primer layer with said top coat composition to form a top coat; (5) curing said primer layer and said top coat thereby making said article or device.

### DETAILED DESCRIPTION

The article or device of the invention has at least one metallic surface. The material for the metallic surface is not particularly limited and can include any metal or metal alloy commonly used in healthcare articles or devices. Exemplary metal material includes stainless steel, tungsten, nickel, cobalt, molybdenum, tin, vanadium, titanium, aluminum, iron, copper, zinc, silver, lead, or the metal alloys thereof. Preferably, the metallic surface contains a biocompatible material such as stainless steel or tungsten.

According to the present invention, the metallic surface is coated with a primer layer and a top coat overlying on the primer layer. By “top coat” herein is meant a layer of coating overlaying the primer layer. As used herein, it is appreciated that one or more additional layers of coating(s) may be disposed over the top coat if desired to form an overcoat for the top coat.

The composition suitable for forming the primer layer contains a silanol-containing organopolysiloxane resin and a crosslinker being alkyl silicate. In one embodiment, the primer composition consists essentially of these two ingredients. Preferably, the silanol-containing organopolysiloxane resin is present in the primer composition in an amount ranging from about 70 to 95 wt %, more preferably from 80 to about 90 wt %, and the crosslinker is present in an amount of from 5 to about 30 wt %, preferably from about 10 to about 20 wt %, all based on the solid content of the primer composition.

The silanol-containing organopolysiloxane resin useful for the primer composition includes silanol terminated organopolysiloxanes. Suitable silanol-containing organopolysiloxanes are represented by Formula I:

MₐD_{b}T_{c}Q_{d}, (Formula I)

where M=R¹R²R³SiO_{1/2}; D=R⁴R⁵SiO_{2/2}; T=R⁶SiO_{3/2}; and Q=SiO_{4/2}, where the subscripts a, b, c, and d are zero or positive and are chosen so that the organopolysiloxane resin has a viscosity, based on a 50 wt % solids solution in an aromatic solvent, varying between about 5 centistokes and 10,000 centistokes, and where each R¹, R², R³, R⁴, R⁵, and R⁶ is independently selected from the group of hydrogen, hydroxyl, C1 to C60 monovalent alkyl radicals; C1 to C60 monovalent halo-alkyl radicals; C1 to C60 monovalent aryl radicals; C1 to C60 monovalent alkylaryl radicals; and C1 to C60 monovalent halo-alkylaryl radicals, subject to the limitation that at least one of R¹, R², and R³ is hydroxyl. In one embodiment, R¹-R⁶ are independently hydroxyl, C1-C40 alkyl radicals, more preferably C1-C20 alkyl radicals, even more preferably C1-C5 radicals, subject to the limitation that at least one of R¹, R², R³ is hydroxyl.

Preferably, the silanol-containing organopolysiloxane resin suitable for the primer composition of the invention has the formula:

DₑT_{f} (Formula II)

wherein D=R⁷R⁸SiO_{2/2}; T=R⁹SiO_{3/2}, where each R⁷, R⁸ and R⁹ is independently alkyl, for example methyl, ethyl or propyl, preferably methyl, hydroxyl, alkoxy or acyloxy, provided that at least one of R⁷, R⁸ and R⁹ per molecule is hydroxyl, e is from about 160 to about 450, and f is from about 15 to about 50. In a preferred embodiment, it is a methyl silicone resin defined by Formula (II).

The crosslinking agent suitable for the primer layer can be alkyl silicates, boric acid, borates, acetoxysilanes, ketoximesilanes, and combinations thereof. In one embodiment, the crosslinking agent is an alkyl silicate. The alkyl silicate used in the present invention is at least one organic solvent-soluble condensate obtained by partially hydrolyzing at least one tetraalkyl silicate represented by the following formula: Si(OR¹⁰)(OR¹¹)(OR¹²)(OR₁₃) (Formula III). In connection with this formula, each occurrence of R¹⁰, R¹¹, R¹², R¹³ is an alkyl group containing 1 to 4 carbon atoms.

Examples of the tetraalkyl silicate include tetramethyl silicate, trimethylmonoethyl silicate, dimethyldiethyl silicate, trimethylmonopropyl silicate, trimethylmonobutyl silicate, monomethyltriethyl silicate, tetraethyl silicate, dimethyldibutyl silicate, triethylmonobutyl silicate, triethylmono-tert-butyl silicate, diethyldibutyl silicate, diethyldi-tert-butyl silicate, tetrapropyl silicate, tetraisopropyl silicate, monoethyltributyl silicate, diisopropyldibutyl silicate, monoisopropyltriisobutyl silicate, monoisopropyl-tri-tert-butyl silicate, tri-sec-butylmono-tert-butyl silicate, tetrabutyl silicate, tetraisobutyl silicate, tetra-sec-butyl silicate, and tetra-tert-butyl silicate. Among these, tetraethyl silicate is especially preferred.

The condensate is a tetraalkyl silicate condensate soluble in organic solvents, which is obtained by partially hydrolyzing the tetraalkyl silicate of Formula (III) in an organic solvent in the presence of a suitable catalyst such as hydrochloric acid using water in an amount required for partial hydrolysis. The especially preferred condensate is a condensate of tetraethyl silicate.

Exemplary borates include but are not limited to trimethylborate, tributylborate, triphenylborate, trihexylborate, tricyclohexylborate.

Suitable acetoxysilanes include vinyltriacetoxysilane and alkylacetoxysilanes such as methyltriacetoxysilane.

Suitable ketoximesilanes are represented by the following general formula: R²⁰ⱼSi(ON═CR²¹₂)₄₋ⱼ, wherein each occurrence of R²° is independently a substituted or unsubstituted monovalent hydrocarbon group of 1 to 10 carbon atoms, each occurrence of R²¹ is independently an unsubstituted monovalent hydrocarbon group of 1 to 10 carbon atoms, and j is equal to 0, 1 or 2.

Illustrative examples for ketoximesilanes include but limited to methyltris(dimethylketoxime)silane, methyltris(methylethylketoxime)silane, ethyltris(methylethylketoxime)silane, methyltris(methylisobutylketoxime)silane, vinyltris(methylethylketoxime)silane, vinyltris(dimethylketoxime)silane, phenyltris(methylethylketoxime)silane, and phenyltris(dimethylketoxime)silane.

If necessary, additional silanes such as methyltrimethoxysilane, methyltriisopropenoxysilane, vinyltrimethoxysilane and vinyltri(2-methoxyethoxy)silane can be used together with the crosslinking agent as specified above.

In a preferred embodiment, solvents are not needed in the primer composition. However, if necessary, solvents may be used in the primer composition. Conventional hydrocarbon solvents such as alkanes, ethers, and the like are useful. Preferably, the solvent has a low boiling point. In one embodiment, the solvent used is hexane.

The primer composition may additionally contain from 5 to 1000 ppm of a catalyst, based on the total weight of the silanol-containing organopolysiloxane and the alkyl silicate in the primer composition. The catalyst promotes the condensation reaction between the components of the primer composition. Suitable catalysts include, but are not limited to, ferric chloride, iron octoate, a carboxylic acid salt of zinc, titanium, tin, zirconium or a combination thereof, such as, for example, zinc 2-ethylhexanoate, zinc octoate, a titanate ester, tetraisopropyltitanate, tetrabutyltitanate, dibutyltin dilaurate, dimethyltin dineodecanoate, dibutyltin dioctoate, dimethyltin oxide, dimethylhydroxytin oleate, dibutyltin bis(acetylacetonate), and zirconium 2-ethylhexanoate. In a preferred embodiment, the catalyst is ferric chloride.

The top coat composition which can be used to form a top coat layer overlaying the primer layer contains an alkenyl functional organopolysiloxane, an organohydrogensiloxane, a hydrosilylation catalyst, and optionally a solvent. The alkenyl functional organopolysiloxane is present in the top coat composition in an amount ranging from about 0.5 to about 30 wt %, preferably from about 1 to about 10 wt % based on the solid content of the top coat composition. The organohydrogensiloxane is present in such an amount that the SiH and Si-alkenyl (Vi) functional groups in the top coat composition are at a molar ratio of from 0.8 to 10 and preferably from 1.2 to 8, more preferably from 1.5 to 5. The hydrosilylation catalyst is present in an amount of from about 5 to about 500 ppm, preferably from about 10 to about 100 ppm, based on the total weight of the alkenyl functional organopolysiloxane and organohydrogensiloxane in the top coat composition.

The alkenyl functional organopolysiloxane suitable for use in the top coat composition can be a vinyl chain-stopped polysiloxane having the general formula:

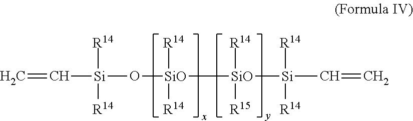

wherein R¹⁴ is a monovalent hydrocarbon radical free of unsaturation, R¹⁵ is a hydrocarbon radical having alkenyl unsaturation, x is from 0 to 6300, y is from 0 to 250, provided that the sum of x+y is at least 80. In one embodiment, R includes substituted or unsubstituted aryl, alkaryl or alkyl groups. Preferably, R¹⁴ is an alkyl, such as methyl, ethyl, propyl preferably a methyl group, and R¹⁵ is a vinyl or a vinyl ether group.

Examples of preferred linear alkenyl functional organopolysiloxanes of the above formula which are suitable for the top coat composition of this invention include (CH₂═CH)(CH₃)₂SiO[Si(CH₃)₂O]ₗ₀₀[Si(CH₃)(CH₂CH₂CH₂CH₂CH═CH₂)O]₂—Si(CH₃)₂(CH═CH₂), (CH₂═CH)(CH₃)₂SiO[Si(CH₃)₂O]₁₀₀[Si(CH₃)(CH═CH₂)O]₂Si(CH₃)₂(CH═CH₂), (CH₂═CH)(CH₃)₂SiO[Si(CH₃)₂O]₆₃₀₀[Si(CH₃)(CH═CH₂)O]₂₅₀Si(CH₃)₂(CH═CH₂), (CH₂═CH)(CH₃)₂SiO[Si(CH₃)₂O]₆₃₀₀[Si(CH₃)(CH═CH₂)O]₆Si(CH₃)₂(CH═CH₂), (CH₂═CH)(CH₃)₂SiO[Si(CH₃)₂O]₈₀Si(CH₃)₂(CH═CH₂), (CH₂═CH)(CH₃)₂SiO[Si(CH₃)(CH═CH₂)O]₁₅₀Si(CH₃)₂(CH═CH₂), and mixtures thereof.

The organohydrogensiloxane of the top coat composition is a substantially linear hydrogen siloxane having a formula selected from the group consisting of

MDᵣD^{H}ₛM

MD^{H}ᵣM

MDᵣD^{H}ₛM^{H}

M^{H}DᵣD^{H}ₛM^{H}, and

M^{H}DᵣM^{H}

where M is defined as R¹⁶₃SiO_{1/2}

M^{H} is defined as H_{g}R¹⁶_{3-g}SiO_{1/2}

D=R¹⁶R¹⁶SiO_{2/2}

D^{H}=R¹⁶HSiO_{2/2}

where each R¹⁶ is independently a monovalent hydrocarbon of from 1 to 40 carbon atoms, preferably from 1 to 20 carbon atoms, more preferably from 1 to 6 carbon atoms and most preferably 1 carbon atom, the subscripts r and s may be zero or positive wherein the sum of r and s ranges from about 10 to about 100, with the proviso that the sum of r and g is at least 2.

In another embodiment of the invention, R¹⁶ includes substituted or unsubstituted aryl, alkaryl or alkyl groups. Preferably, R¹⁶ is an alkyl, such as a methyl group.

In a preferred embodiment, the organohydrogensiloxane is MD^{H}ₖM, where k is greater than 0 and less than about 100, preferably from 1 to 50, more preferably from 3 to 25 and most preferably from 5 to 10. M and D^{H} are as defined above.

Conventional platinum hydrosilylation catalysts may be used as a hydrosilylation catalyst, catalyzing the addition reaction between the carbon-carbon multiple bond in the alkenyl functional polyorganosiloxane described above and the silicon-bonded hydrogen atoms (—SiH) in the organohydrogensiloxane crosslinking agent of the invention. In general, any hydrosilylation catalyst for addition-crosslinking silicone compositions may be used. Those preferably used are metal-containing catalysts, such as platinum, palladium, iridium, rhodium and ruthenium, with preference given to platinum and platinum compounds. Particular preference is given to polyorganosiloxane-soluble platinum-vinylsiloxane complexes and hexachloroplatinic acid.

Optionally, the top coat composition contains an inhibitor for the hydrolsilylation metal catalysts. The inhibitors are well known in the organosilicon art. Examples of various classes of such metal catalyst inhibitors include unsaturated organic compounds such as ethylenically or aromatically unsaturated amides, U.S. Pat. No. 4,337,332; acetylenic compounds, U.S. Pat. Nos. 3,445,420; 4,347,346 and 5,506,289; ethylenically unsaturated isocyanates, U.S. Pat. No. 3,882,083; olefinic siloxanes, U.S. Pat. No. 3,989,667; unsaturated hydrocarbon diesters, U.S. Pat. Nos. 4,256,870; 4,476,166 and 4,562,096, and conjugated ene-ynes. U.S. Pat. Nos. 4,465,818 and 4,472,563; other organic compounds such as hydroperoxides, U.S. Pat. No. 4,061,609; ketones, U.S. Pat. No. 3,418,731; sulfoxides, amines, phosphines, phosphites, nitriles, U.S. Pat. No. 3,344,111; diaziridines, U.S. Pat. No. 4,043,977; half esters and half amides, U.S. Pat. No. 4,533,575; and various salts, such as U.S. Pat. No. 3,461,185. It is believed that the compositions of this invention can comprise an inhibitor from any of these classes of inhibitors.

The inhibitors may be selected from the group consisting of ethylenically unsaturated amides, aromatically unsaturated amides, acetylenic compounds, ethylenically unsaturated isocyanates, olefinic siloxanes, unsaturated hydrocarbon diesters, unsaturated hydrocarbon mono-esters of unsaturated acids, conjugated ene-ynes, hydroperoxides, ketones, sulfoxides, amines, phosphines, phosphites, nitriles, and diaziridines.

Optionally, solvents can be used in the top coat compositions. In a preferred embodiment, the top coat composition includes from about 70 to about 99.5 wt %, preferably from about 80 to about 95 wt % of an inert solvent. Suitable solvents include alkyl or aromatic hydrocarbons or mixtures thereof. Exemplary solvents include but are not limited to heptanes, hexanes, and xylenes. Optionally, ethers and alcohols can be included in minor amounts, usually no more than 25% of the solvent system. Exemplary alcohols include methanol, ethanol, propanol, isopropanol, n-butanol, tert-butanol, methoxypropanol, ethylene glycol, diethylene glycol butyl ether, or combinations thereof. Other water miscible organic solvents such as acetone, methyl ethyl ketone, ethylene glycol monopropyl ether, and 2-butoxy ethanol, can also be utilized in minor amounts if desired.

Both the primer composition and the top coat composition can be made by simply mixing the components described above. Once the primer and the top coat compositions are formed, they can be applied to the metallic surface of an article or device.

According to another embodiment of the invention, there is provided a method for making an article or a device having a coated metallic surface comprising the steps of: (1) providing an article or a device having at least one metallic surface; (2) providing a primer composition and a coating composition according to the present invention; (3) contacting said metallic surface with said primer composition to form a primer layer; (4) contacting said primer layer with said top coat composition to form a top coat; (5) curing said primer layer and said top coat thereby making said article or device.

The primer composition can be applied to the metallic surface employing techniques known to one skilled in the art, e.g. by dipping, brushing, wiping, spraying, total immersion, etc., with dipping and spraying being the preferred techniques. If the primer composition contains a solvent, it is preferable to evaporate the solvent first before applying the top coat composition to the substrate.

The top coat compositions can be applied to the primed metallic surface also by techniques such as dipping, brushing, wiping, spraying, total immersion etc. Preferably, needles are dipped into the top coat composition to form a top coat on the needle.

Curing of the primer and the top coat can be accomplished by conventional methods well known in the art, for example, heat curing via oven. Suitable curing conditions are dependent on the coating compositions. A preferred curing condition is to heat the coated substrate at 80 to 300° C. for one or two seconds to about 20 minutes. The more preferred curing condition is to heat the coated substrate at 80 to 200° C. for about 5 seconds to about 5 minutes.

The coated article or device of the invention can be used in many applications. Since the coatings on the articles are durable and exhibit low coefficient of friction, the coatings are particularly useful in healthcare related articles or devices for example surgical devices, needles, preferably surgical needles, razors, and cutting/slicing devices. Applying this coating to devices such as surgical needles can help improve the ability of the device to pass through tissue with less force and are therefore preferred embodiments.

The following examples are illustrative and not to be construed as limiting of the invention as disclosed and claimed herein. All parts and percentages are by weight and all temperatures are degrees Celsius unless explicitly stated otherwise. All patent applications, patents and other publications cited herein are incorporated by reference in their entirety.

### EXAMPLES

### Examples 1-7 and Comparative Examples (a)-(g)

### Coating of Stainless Steel Panels

Part I: Preparation of a Primer Composition

Components having the following structures were mixed to form a primer composition: 84.23 wt % of (T_{89.5}D₉)ₓOH, x≃2-5 based on 0.05 wt % OH content, 15.70 wt % of T(OCH₂CH₃)_{˜2.5}, and 725 ppm ferric chloride.

Part II: Preparation of a top coat composition

The following components were mixed to form a top coat composition: 96.67 parts of hexane, 3.33 parts of a vinyl functional polydimethylsiloxane gums (48.34 parts of M^{vi}D₆₃₀₀D^{vi}₂₅₀M^{vi} plus 51.66 parts of MᵥᵢD₆₃₀₀D^{vi}₆M^{vi}), 0.033 parts of crosslinker M^{vi}D1₁₅₀M^{vi} and 0.028 parts of platinum catalyst.

Part III: Preparation of Coated Metallic Substrate

A set of 14 stainless steel panels (Q Panel RS-14, 0.06′×1′×4′) were cleaned. The primer composition prepared in part I was wiped on seven panels with a clean cloth, then air dried for ten minutes.

Seven primed panels Examples 1-7 and remaining seven unprimed panels Comparative Examples (a)-(g) were coated with the top coat composition described in part II using dip coating technique.

Coated panels were than placed in an air forced oven set at 190° C. and allowed to cure for 2, 2.5, 3, 3.5, 4, 4.5, and 5 minutes. They were then removed from the oven and allowed to cool down to room temperature. Kinetic coefficients of friction (CoF) against polyurethane substrate of each coating were measured and the results were shown at Table 1. SP-101B Slip/peel Tester from Instrumentors, Inc. was used to perform the CoF testing according to ASTM Standard D-1894. From the results, it can be seen that the presence of a primer layer helps to reduce the coefficient of friction of the coatings. For example, as shown in example 7 and comparative example (g), the kinetic coefficient of friction was reduced from 0.027 to 0.009 when the metallic panel was primed as compared to unprimed substrate.

TABLE 1

| | Coefficient of Friction (kinetic) | |
|---|---|---|
| Cure time | Comparative | Examples (E) |
| @ 190° C. | Examples (CE) | Primer and |
| (minutes) | Top coat only | top coat |

| | | | | |
|---|---|---|---|---|
| 2 | CE (a) | 0.094 | E1 | 0.066 |
| 2.5 | CE (b) | 0.094 | E2 | 0.033 |
| 3 | CE (c) | 0.08 | E3 | 0.048 |
| 3.5 | CE (d) | 0.082 | E4 | 0.044 |
| 4 | CE (e) | 0.078 | E5 | 0.033 |
| 4.5 | CE (f) | 0.072 | E6 | 0.025 |
| 5 | CE (g) | 0.027 | E7 | 0.009 |

### Examples 8-10 and Comparative Examples (h)-(j)

### Coating of Tungsten Plates

A set of six polished tungsten plates were thoroughly cleaned with trichloroethylene solvent and allowed to dry. Three of the plates were coated with the primer composition prepared in part I, Example 1 by first wiping the primer composition on the plates with a lint free cloth, then allowing the primer composition to dry for five minutes. Three of the remaining plates were unprimed.

Next, the three primed and three unprimed plates were coated with the top coat composition described in part II, Example 1 using dip coating technique.

The coated plates were divided into three sets, each set containing one primed and one unprimed plates. The first set of the coated plates, Example 8 and Comparative Example (h), were cured at 200° C. for 90 seconds; the second set of the coated plates, Example 9 and Comparative Example (i), were cured at 185° C. for 120 seconds; and the third set of the coated plates, Example 10 and Comparative Example (j), were cured at 200° C. for 120 seconds. All the cured coated plates were allowed to cool down to room temperature.

After each plate in the first set was rubbed with a finger with significant amount of pressure, the first set plates were tested for release value according to the following procedure: one inch wide Tesa7475 standard acrylic adhesive tape manufactured by Tesa Germany was applied to each plate; the tape was rolled with the mechanical 4.5 pounds rubber roller at 12 inches per minute to remove air and assure uniform contact of adhesive with coated surface; the tape was then stripped from the plate at 12 inches per minute speed, 180 degrees angle; and the force needed to strip the tape was measured on TMI Release and Adhesion Tester. The results are shown in Table 2 below.

Finger rub off tests were applied to half of the coated area on each plate in the second and third sets. The other half of the coated area on each plate were untouched. Rubbed area and un-rubbed area on each plate was then tested separately for release value according to the procedure described above. The results are shown in Table 2 below.

TABLE 2

| Cure Time (seconds)/ | Release Value (grams/inch) | |
|---|---|---|
| Temperature (° C.) | Rubbed Area | Un-rubbed area |

| | | | | |
|---|---|---|---|---|
| 90/200 | E8 | 422 | | |
| | C(h) | 1452 | | |
| 120/185 | E9 | 903 | E9 | 20 |
| | C(i) | 1665 | C(i) | 48 |
| 120/200 | E(10) | 125 | E(10) | 10 |
| | C(j) | 1297 | C(j) | 22 |

Lower release values typically indicate good coating coverage, and an increase in release value after rubbing indicates loss of coverage. The results in Table 2 indicate that a primer composition according to the invention significantly improves anchorage of top coat. For example, as compared to unprimed comparative examples (h)-(j), primed examples 8-10 show a significantly lower release of the acrylic tape off tungsten plates after the plates were exposed to the rub off test. Further, although the release values increased for both primed and unprimed plates after they were rubbed, the differences between un-rubbed and rubbed areas for primed examples 8-10 are significantly lower than that for unprimed comparative examples (h)-(j).

While the invention has been described above with references to specific embodiments thereof, it is apparent that many changes, modifications and variations can be made without departing from the inventive concept disclosed herein. Accordingly, it is intended to embrace all such changes, modifications and variations that fall within the spirit and broad scope of the appended claims.

1. An article comprising a metallic surface, a primer layer disposed on said metallic surface, and a top coat disposed on said primer layer, wherein said primer layer is formed from a primer composition containing a silanol-containing organopolysiloxane resin and a crosslinking agent selected from the group consisting of alkyl silicates, boric acid, borates, acetoxysilanes, ketoximesilanes, and combinations thereof, and wherein said top coat is formed from a top coat composition containing an alkenyl functional polyorganosiloxane, an organohydrogensiloxane, and a hydrosilylation catalyst.

2. The article of claim 1 wherein said metallic surface comprises a metal selected from the group consisting of stainless steel, tungsten, nickel, cobalt, molybdenum, tin, vanadium, titanium, aluminum, iron, copper, zinc, silver, lead, and combinations thereof.

3. The article of claim 2 wherein said metallic surface is made of stainless steel.

4. The article of claim 2 wherein said metallic surface contains tungsten.

5. The article of claim 1 wherein said silanol-containing organopolysiloxane resin in the primer composition is represented by the following formula:
MₐD_{b}T_{c}Q_{d}, (Formula I)
wherein M=R¹R²R³SiO_{1/2}; D=R⁴R⁵SiO_{2/2}; T=R⁶SiO_{3/2}; and Q=SiO_{4/2}, wherein the subscripts a, b, c, and d are zero or positive and are chosen so that the resin has a viscosity, based on a 50 wt % solids solution in an aromatic solvent, varying between about 5 centistokes and 10,000 centistokes, and where each R¹, R², R³, R⁴, R⁵, and R⁶ is independently selected from the group of hydrogen, hydroxyl, C1 to C60 monovalent alkyl radicals; C1 to C60 monovalent halo-alkyl radicals; C1 to C60 monovalent aryl radicals; C1 to C60 monovalent alkylaryl radicals; and C1 to C60 monovalent halo-alkylaryl radicals, subject to the limitation that at least one of R¹, R², and R³ is hydroxyl.

6. The article of claim 5 wherein said silanol-containing organopolysiloxane is represented by Formula (II):
DₑT_{f} (Formula II)
wherein D=R⁷R⁸SiO_{2/2}; T=R⁹SiO_{3/2}, wherein each R⁷, R⁸and R⁹ is independently aklyl, hydroxyl, alkoxy or acyloxy, provided that at least one of R⁷, R⁸ and R⁹ per molecule is hydroxyl, e is from about 160 to about 450, and f is from about 15 to about 50.

7. The article of claim 1 wherein said alkyl silicate in said primer composition is a condensate obtained by partially hydrolyzing at least one tetraalkyl silicate represented by the following formula: Si(OR¹⁰)(OR¹¹)(OR¹²)(OR¹³), wherein each occurrence of R¹⁰, R¹¹, R¹² and R¹³, represents an alkyl group containing 1 to 4 carbon atoms.

8. The article of claim 7 wherein said alkyl silicate is a condensate of tetraethyl silicate.

9. The article of claim 1 wherein said primer composition additionally contains a condensation catalyst.

10. The article of claim 9 wherein said catalyst is ferric chloride.

11. The article of claim 1 wherein said alkenyl functional organopolysiloxane is a vinyl chain-stopped polysiloxane having the general formula: 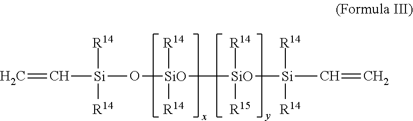
wherein R¹⁴ is a monovalent hydrocarbon radical free of unsaturation, R¹⁵ is a hydrocarbon radical having alkenyl unsaturation, x is from 0 to 6300, y is from 0 to 250, provided that the sum of x+y is at least 80.

12. The article of claim 11, wherein R¹⁴ is methyl, and R¹⁵ is vinyl or vinyl ether.

13. The article of claim 1 wherein the organohydrogensiloxane is a substantially linear hydrogen siloxane having a formula selected from the group consisting of
MDᵣD^{H}ₛM
MD^{H}ᵣM
MDᵣD^{H}ₛM^{H}
M^{H}DᵣD^{H}ₛM^{H}, and
M^{H}DᵣM^{H}
where M is defined as R¹⁶₃SiO_{1/2}
M^{H} is defined as H_{g}R¹⁶_{3-g}SiO_{1/2}
D=R16R¹⁶SiO_{2/2}
D^{H}=R¹⁶HSiO_{2/2}
where each R¹⁶ is independently a monovalent hydrocarbon of from 1 to 40 carbon atoms, the subscripts r and s may be zero or positive wherein the sum of r and s ranges from about 10 to about 100, with the proviso that the sum of r and g is at least 2.

14. The article of claim 13 wherein the organohydrogensiloxane is MD^{H}ₖM, where f is greater than 0 and less than about 100, M and D^{H} are as defined in claim 12.

15. The article of claim 1 wherein said hydrosilylation catalyst is a platinum based complex.

16. The article of claim 1 wherein said top coat composition contains a solvent.

17. The article of claim 1 wherein the article is a surgical needle.

18. The article of claim 1 wherein the article is a surgical device.

19. A method for making an article or a device having a coated metallic surface comprising the steps of: (1) providing an article or a device having at least one metallic surface; (2) providing a primer composition and a coating composition according to claim 1; (3) contacting said metallic surface with said primer composition to form a primer layer; (4) contacting said primer layer with said top coat composition to form a top coat; (5) curing said primer layer and said top coat thereby making said article or device.

20. The method of claim 19 wherein the step of contacting the metallic surface with the primer composition is selected from the group consisting of dipping, spraying, brushing or wiping.

21. The method of claim 19 wherein said method additionally comprises a step of drying the primer composition to form a primer layer.

22. The method of claim 19 wherein said primer layer and said top coat are cured at a temperature of from 80 to 300° C. for about one second to twenty minutes.

23. An article or device prepared by the method of claim 19.
